(19) United States
(12) Patent Application Publication
(10) Pub. No.: US 2003/0147981 A1
(43) Pub. Date: Aug. 7, 2003

(54) Method of determining a dosage of anti-oxidant for an individual

(76) Inventor: Jan Gillam, Camberwell (AU)

Correspondence Address: GREER, BURNS & CRAIN, 300 S WACKER DR, 25TH FLOOR, CHICAGO, IL, 60606, US

(21) Appl. No.: 10/301,908

(22) Filed: Nov. 22, 2002

Related U.S. Application Data
(63) Continuation-in-part of application No. PCT/AU01/00597, filed on May 23, 2001.

(30) Foreign Application Priority Data
May 23, 2000 (AU) PO 7803

(51) Int. Cl. A61K 35/78; A61K 31/385; A61K 31/355

(52) U.S. Cl. 424/770; 514/440; 514/458; 514/474

(57) ABSTRACT

A method of determining a dosage of anti-oxidant for an individual person, wherein the dosage is determined on the basis of an individual factor and a stress index. The individual factor is based on a weight factor of the individual, an age factor of the individual and a training factor of the individual. The training factor is based on training history of the individual, and the stress index is based on current and future physical activity of the individual. A formulation based on this method is also described.

### RELATED APPLICATION

This is a Continuation in Part of International Application PCT/AU01/00597, having an International Filing Date of May 23, 2001.

### BACKGROUND

This invention relates to a method of determining a dosage of anti-oxidant for an individual person and more particularly, but not exclusively, to a method of determining a dosage of anti-oxidant for an athlete, and a formulation utilizing this method.

Molecular oxygen is essential in the production of energy that our bodies need in order to perform aerobic exercise. This process, which occurs within the mitochondria of the cell, involves oxygen accepting up to four additional electrons, and is called an oxidation reaction. However, when molecular oxygen only accepts between one and three electrons, a variety of oxygen free radicals (called superoxide, peroxide or hydroxy radicals) are formed. Because oxygen is only partly oxidized these free radicals are extremely reactive owing to oxygen being only partly oxidized. It is estimated that for every 100 oxygen molecules involved in oxidative metabolism, approximately four of them form oxygen radicals.

Anti-oxidants are chemical molecules, present in small amounts in the body that can accept an electron from an oxygen radical, thus deactivating it, and preventing oxidative damage. The body produces its own anti-oxidants, the most important of which is glutathione or GSH. The body also produces four anti-oxidant enzymes (superoxide dismutase, catalase, glutathione peroxidase and glutathione reductase), which can detoxify the oxygen radicals to harmless molecules such as water. These anti-oxidant enzymes require the mineral cofactors, selenium, copper, zinc, iron and manganese to function effectively. Selenium is of particular importance in some parts of the world as many of the soils are selenium deficient.

Vitamins E, C and A have long been recognized as important anti-oxidants obtained from our diet. In addition, a diverse group of compounds called flavonoids are found in many plants, fruits and vegetables. Flavonoids including oligomeric proanthocyanidins (OPC's) are now recognized as a key source of dietary anti-oxidants and play an important role in human health. Particularly high concentrations of flavonoids and OPC's are found in the bark of Pinus radiata trees. Bioflavonoids are extracted from the bark of young New Zealand radiata pine trees using only pure water in a water extraction process. Such bioflavonoids are a potent source of OPC's and other important natural dietary anti-oxidants. Another anti-oxidant, alpha lipoic acid, has recently been found to increase the levels of GSH inside the cell, thus increasing the body's protection against oxidative damage.

When the number of oxygen free radicals within the body increases beyond the amount of anti-oxidants in the body, the body is said to be under ‘oxidative stress’. These oxygen radicals rapidly react with fats, proteins and DNA, damaging their molecular structure, which can cause abnormal metabolic and cellular functions, disruptions in cell structure, leakage of essential enzymes involved in energy production and genetic damage that may lead to the development of chronic diseases, such as cancer, later in life.

Intense exercise increases aerobic metabolism (and hence oxygen radical production) by up to 20 times compared with normal resting conditions. This means that the level of oxidative stress experienced by the body is increased in proportion to the exercise intensity. The blood levels of GSH rapidly decrease in response to moderate intensity exercise, and as a consequence oxidative damage increases. Recent research has shown that the use of high-potency anti-oxidant supplements can significantly reduce measures of muscle, blood cell and tissue damage in athletes and active individuals by at least 25%. An increase in the total anti-oxidant capacity resulting from regular physical activity may also be responsible for the reduced muscle fatigue and improvements in physical performance. It has recently been suggested that all active individuals should take anti-oxidant supplements to reduce the likelihood of developing many diseases shown to be associated with oxidative degeneration.

As recovery from exercise is a major concern for athletes in heavy training, a reduction in the level of tissue damage means a faster recovery for the athlete. In addition, research has also shown that anti-oxidant supplements increase the plasma testosterone to cortisol ratio during the post-exercise recovery period, thus assisting muscle repair regeneration.

The important role that dietary anti-oxidants can play on the health and performance of athletes is only now becoming recognized. Anti-oxidants have been shown to reduce muscle and tissue damage and to help prevent the onset of degenerative diseases.

Anti-oxidants can also play a role in improving the health, fitness and well-being of people living in the modern, high-stressed, fast-paced world.

However, the dosage of anti-oxidants is typically haphazard, and it would be beneficial for there to be a systematic method of determining a dosage of anti-oxidants according to the body's requirements.

### SUMMARY

In accordance with one aspect of the present invention, there is provided a method of determining a dosage of anti-oxidant for an individual person, wherein the dosage is determined on the basis of an individual factor and a stress index, the individual factor being based on a weight factor of the individual, an age factor of the individual and a training factor of the individual based on training history of the individual, and the stress index being based on current and/or future physical activity of the individual.

In accordance with another aspect of the present invention, there is provided a method of determining a dosage of anti-oxidant for an individual person, wherein the dosage is determined on the basis of an individual factor (IF) and a stress index (SI),

IF=WF+AF+TF,

SI=Sum of {PATF×TIF×TTF} for each possible PATF/TIF combination, and the dosage increases as each of IF and SI increases;

wherein

WF is the weight factor which increases with increasing weight of the individual,

AF is the age factor which increases with increasing age of the individual,

TF is the training factor which increases as the extent of past physical training of the individual decreases,

PATF is a physical activity type factor of a current and/or future physical activity of the individual and PATF increases as the physical exertion required to perform the corresponding physical activity increases,

TIF is the training intensity factor, where TIF increases as the intensity of the individual's performance of a corresponding physical activity increases, and TTF is a training time factor for a corresponding PATF/TIF combination, where

TTF increases as the time spent by the individual performing a corresponding physical activity increases.

In accordance with another aspect of the present invention, there is provided an anti-oxidant mixture comprising the following amounts of the following constituents: between 100c and 450c mg of bioflavonoids extracted from pine bark extract, between 50c and 150c mg of Alpha lipoic acid, between 50c and 300c mg of Vitamin C and/or its derivatives, and between 100c and 450c mg of Vitamin E and/or its derivatives, wherein c is a positive number.

Preferable the formulation also includes low levels of copper, manganese, selenium and zinc.

This ensures that adequate amounts of these minerals are available to provide the essential co-factors for the four intrinsic anti-oxidants enzymes systems.

The formulation is thus specifically designed to combat the formation of oxygen radicals in all three targeted cellular components; cytosol; lipids; and cell membranes.

### DETAILED DESCRIPTION

The invention will now be further described with reference to more detailed examples.

The applicant has determined that the type, intensity, duration and frequency of an individual's training determines the individual's level of oxidative stress, and thus his or her oxidative stress index (OSI). The greater the OSI, the greater the dose of anti-oxidant required to detoxify the oxygen radicals generated. In addition, factors such as age, weight, gender and whether the individual is trained or untrained will determine his or her body's individual anti-oxidant capacity.

a) Exercise Type

Since oxygen radicals are produced as a by-product of aerobic metabolism, strenuous aerobic exercise requires a greater level of anti-oxidant protection than does exercise that relies more on anaerobic metabolism, muscular strength and power. Many field and court sports such as rugby or tennis are a combination of aerobic and anaerobic energy production, and still require anti-oxidant supplementation. Studies have shown significantly lower plasma vitamin E and C levels in elite swimmers compared to basketballers and gymnasts, which is indicative of the higher level of oxidative stress in endurance athlete groups.

b) Exercise Intensity

The higher the exercise intensity, especially during aerobic exercise, the greater the oxygen consumption, which means higher levels of oxidative stress are placed on the active tissues of the body. Research has demonstrated that increased exercise intensity results in greater levels of oxidative stress and damage.

c) Exercise Duration and Training Frequency

The longer the exercise duration and the greater the training frequency, the greater the numbers of oxygen radicals produced within the body. Increased training volumes, particularly in those sports that are aerobically based, have been shown to cause depletion of plasma and tissue anti-oxidants.

d) Age and Gender

As we age, our anti-oxidant capacity and hence our ability to cope with increasing levels of oxidative stress declines. The levels of plasma GSH progressively decrease from 25 to 45 years of age to 50% of their original level, irrespective of the state of training. As a consequence the concentration of lipid peroxides, an index of oxidative damage to lipids, rises with increased age.

In addition, the capacity of the anti-oxidant enzymes has been shown to decrease with increasing age. This means that there is an increased need for supplemental anti-oxidant protection as we age, especially when undertaking strenuous exercise.

The female sex hormone, estrogen, has been shown to possess anti-oxidant activity, so women in their reproductive years (ie. prior to menopause at around 50 years), have a lower requirement for exogenous anti-oxidants than men of similar age. In other words, females of similar age tend to have lower levels of oxidative damage than males. However, after menopause, women have similar anti-oxidant requirements to men.

e) Body Weight

Individuals with greater body (and muscle) mass need a proportionally greater dosage of anti-oxidant. This ensures that all metabolically active tissues are provided with adequate levels of anti-oxidant protection.

f) Training Level

As athletes undertake training to increase their aerobic capacity, the capacity of their anti-oxidant enzyme systems also increases. Consequently the ability to protect their metabolically active tissues against the oxidative stress produced during training is increased. As the irregularly active individual does not achieve these adaptations, the use of anti-oxidant supplements is probably even more important for untrained individuals. Despite these enzymatic adaptations, the level of residual oxidative damage is still present in trained athletes at rest.

Elite endurance athletes undertaking periods of heavy training have an increased susceptibility to ‘overtraining’. This syndrome is characterized by impaired physical performance, prolonged periods of fatigue, increased levels of muscle damage and soreness, hormonal disturbances and impaired immune function. Anti-oxidant supplementation has been shown to significantly reverse a hormonal indicator of overtraining, enhance immune function and reduce the frequency of infective episodes following exercise. The anti-inflammatory properties of anti-oxidants may also reduce chronic muscle soreness associated with heavy training.

The applicant has determined a method of determining a dosage of anti-oxidant for an individual, based on the above factors. A preferred embodiment of the invention will now be described, by way of example only, with reference to the tables and equations incorporated herein.

In accordance with a preferred embodiment of the present invention, there is provided a method of determining a dosage of anti-oxidant for an individual person, including the following steps.

Step 1: Determine an Individual Factor (IF)

To determine the individual factor based on an individual's age, gender and body weight, the following equation is used:

Individual Factor (IF)=Age Factor+Weight Factor+Training Factor,

where the Age Factor, Weight Factor and Training Factor are determined as follows.

TABLE 1
(i) Age Factor (AF)

| | | | |
|---|---|---|---|
| Male | less than or equal to 30 | 31 to 45 | greater than or equal to 45 |
| | years | years | years |
| Female | less than or equal to 35 | 36 to 55 | greater than or equal to 55 |
| | years | years | years |
| Age | 1 | 1.5 | 2 |
| Factor | | | |

TABLE 2
(ii) Body Weight Factor (WF)

| | | | |
|---|---|---|---|
| Weight | less than or equal to | 60 kg to | greater than or equal to |
| | 60 kg | 90 kg | 90 kg |
| Weight | 1 | 1.25 | 1.5 |
| Factor | | | |

(iii) Training Factor (TF)

Whether the individual is “trained” or “untrained” has an important bearing on the capacity of the individual's anti-oxidant enzymes to detoxify oxygen free radicals.

The following guidelines are provided to determine whether the individual is classified as “trained” or “untrained”.

If, over the past 3 months, the individual has been exercising aerobically at least 5 times per week for at least 30 minutes at a moderate intensity level, the individual is considered to be “trained’. Alternatively, other tests may be used, for example, if the individual raises a significant sweat response in mild conditions (20-25° C.), he or she may be considered “trained”.

If the individual is ‘trained’, he or she has a Training Factor of 0. If the individual is “untrained”, the individual has a Training Factor of 0.5.

It is now possible to calculate the Individual Factor of the individual by adding together the Age Factor, the Body Weight Factor, and the Training Factor.

### EXAMPLE

Individual Factor (IF)=Age Factor+Weight Factor+Training Factor

As an example, consider a case in which the individual is an untrained male, 40 years of age, with a body weight of 85 kg.

The Individual Factor of the individual is calculated as:

IF=1.5+1.25+0.5=3.25,

where the components added are determined by sections (i) to (iii), above.

Step 2: Determine the Oxidative Stress Index (OSI)

The type, intensity, duration and frequency of the individual's training determines his or her level of oxidative stress. The OSI is based on the individual's training load and is determined by the following equation:

$$OSI = \frac{\begin{matrix}Sum \{ Physical Activity Type Fa ctor ( PATF ) \times \\ Exercise Load Factor ( ELF ) \} ,\end{matrix} }{10}$$

where 10 is an arbitrary scaling factor to get the OSI values within a desired range to facilitate presentation in a convenient form (see for example Table 8, below).

Calculation tables such as those in Tables 5 to 7 may be provided to assist in performing this calculation.

(i) Physical Activity Type Factor (PATF)

Consider the main physical activities of the individual (either while training or competing) and allocate a “physical activity type factor” (ie. a number from 1 to 3) from Table 3, below, for each physical activity type. Different activities may be written into tables, such as those in Tables 5 to 7, in order of the “physical activity type factor”.

TABLE 3

| | Physical | |
|---|---|---|
| Physical Activity | Activity | |
| Classification | Type Factor | Typical Physical Activities |
| Heavy endurance | 3 | Triathlon and ultra-endurance sports, |
| sports | | distance and cross-country running, |
| | | swimming, road cycling, cross- |
| | | country skiing, rowing and paddling |
| Combination | 2 | Alpine skiing, track cycl- |
| aerobic & | | ing, ice-skating, soccer, rugby, touch |
| anaerobic sports, | | rugby, Australina rules for football, |
| field games & | | basketball, squash, tennis, badminton, |
| court sports, | | table tennis, boxing, wrestling, judo |
| combat sports | | |
| Low intensity | 1 | Golf, fencing, sprinting and field |
| aerobic strength & | | sports in track and field, anaerobic |
| power, sports | | sports, weight training and weight |
| skills, target and | | lifting, bodybuilding, gymnastics, |
| artistic sports | | diving, shooting, archery |

(ii) Exercise Load Factor (ELF)

To determine the exercise load factor of the individual for each physical activity type, the following formula may be used, and is based on the various training types and the number of hours spent at each during a typical training week.

Exercise Load Factor={Training hours per week (TTF)×Training Intensity Factor (TIF)}

The training intensity for each training type is estimated and is assigned a Training Intensity Factor as follows. The number of hours spent by the individual doing each physical activity each week is also estimated. These values are entered into tables, such as those in Tables 5 to 7.

TABLE 4

| | Training Intensity |
|---|---|
| | Factor |
| Easy or low intensity | 1 |
| Moderately hard training or moderate | 2 |
| intensity training | |
| Hard, high intensity or near maximal | 3 |
| training | |

### EXAMPLE

Consider the case in which the individual is a distance runner (IF=3.25) who completes an average of 12 hours in total per week, (average of 2 hours of training per day, 6 days per week), the 12 hours being spent as follows: 3 hours per week Race/Pace and Hill Training at a moderate intensity; 6 hours per week Slow Distance running at a low intensity; and 3 hours per week Interval Training at near maximum intensity. The individual also performs moderate intensity weight training in two sessions per week for one hour per session. The OSI would be calculated as follows:

TABLE 5

| | Weight training at near moderate | |
|---|---|---|
| PATF 1 Activities | intensity | OSI₁ |
| Intensity Factor | 2 | |
| Hours spent per week | 2 | |
| ELF (Intensity factor × | 4 | |
| hours) | | |
| | 1 × ELF = 1 × 4 | 4 |

TABLE 6

| | The individual performs no PATF | |
|---|---|---|
| PATF 2 Activities | 2 Activities | OSI₂ |
| Intensity Factor | 0 | |
| Hours spent per week | 0 | |
| ELF (Intensity factor × | 0 | |
| hours) | | |
| | 2 × ELF = 2 × 0 | 0 |

TABLE 7

| | Race/pace | Slow | Interval training | |
|---|---|---|---|---|
| PATF 3 | and hill | distance | at near maximum | |
| Activities | training | running | intensity | OSI₃ |
| Intensity Factor | 2 | 1 | 3 | |
| Hours spent per | 3 | 6 | 3 | |
| week | | | | |
| ELF (Intensity | 6 | 6 | 9 | |
| factor × hours) | | | | |
| | 3 × ELF = 3 × | | | 63 |
| | (6 + 6 + 9) = 3 × | | | |
| | 21 | | | |

$$OSI = \frac{\begin{matrix}Sum \{ Physical Activity Type Factor ( PATF ) \times \\ Exercise Load Factor ( ELF ) \}\end{matrix}}{10} ⇒ OSI = \frac{{OSI}_{1} + {OSI}_{2} + {OSI}_{3}}{10} ⇒ OSI = \frac{4 + 0 + 63}{10} = \frac{67}{10} = 6.7$$

Step 3: Determine the dosage of anti-oxidant using the Individual Factor (IF) and the Oxidative Stress Index (OSI)

Using the Individual Factor (IF) of the individual and the Oxidative Stress Index (OSI) of the individual, the dosage of anti-oxidant for the individual can be determined by using the nomogram shown in Table 8, below.

TABLE 8

| OSI Rating | | Number of capsules of anti-oxidant per day | | | | | | | | |
|---|---|---|---|---|---|---|---|---|---|---|
| Extreme | 10 | 3 | 3 | 3 | 3 | | | | | |
| Extreme | 9 | 3 | 3 | 3 | 3 | 3 | | | | |
| Very Heavy | 8 | 2 | 3 | 3 | 3 | 3 | 3 | | | |
| Very heavy | 8 | 2 | 2 | 3 | 3 | 3 | 3 | 3 | | |
| Heavy | 6 | 2 | 2 | 2 | 3 | 3 | 3 | 3 | 3 | |
| Heavy | 5 | 2 | 2 | 2 | 2 | 3 | 3 | 3 | 3 | 3 |
| Moderate | 4 | 1 | 2 | 2 | 2 | 2 | 3 | 3 | 3 | 3 |
| Moderate | 3 | 1 | 1 | 2 | 2 | 2 | 2 | 3 | 3 | 3 |
| Light | 2 | 1 | 1 | 1 | 2 | 2 | 2 | 2 | 3 | 3 |
| Light | 1 | 1 | 1 | 1 | 1 | 2 | 2 | 2 | 2 | 3 |
| Individual Factor | | 2.00 | 2.25 | 2.50 | 2.75 | 3.00 | 3.25 | 3.50 | 3.75 | 4.00 |

As an example of a dosage calculation, consider an individual with an IF of 3.25 and an OSI of 6.7. By finding the dosage in the nomogram corresponding to these IF and OSI values, it can be determined that the dosage of anti-oxidant for the individual is 3 capsules per day.

Numbers inside the nomogram represent the number of capsules per day recommended to be taken of an anti-oxidant, where each capsule comprises the following amounts of the respective constituents:

150 mg of bioflavonoids extracted from pine bark extract;

50 mg of Alpha lipoic acid;

100 mg of a Vitamin C ester; and

150 mg of Vitamin E as d-alpha—tocopherol acid succinate.

It should be noted that in place of or in addition to the above Vitamin C ester, each capsule may contain Vitamin C and/or its derivatives, for example sodium, calcium, magnesium and potassium salts of Vitamin C and Vitamin C esters.

It should also be noted that in place of or in addition to the above Vitamin E as d-alpha tocopherol acid succinate, each capsule may contain Vitamin E and/or its derivatives, for example d-alpha tocopheryl acid succinate and d-alpha tocopheryl acid acetate.

It is recommended that:

when taking 3 capsules per day, one capsule is taken with water 10 to 20 minutes before each meal;

when taking 2 capsules per day, one capsule is taken with water 10 to 20 minutes before morning and evening meals; and

when taking 1 capsule per day, the one capsule is taken with water 10 to 20 minutes before the morning meal.

While a particular embodiment of the Method of Determining a Dosage of Anti-Oxidant for an Individual has been described herein, it will be appreciated by those skilled in the art that changes and modifications may be made thereto without departing from the invention in its broader aspects and as set forth in the following claims.

What is claimed:

1. A method of determining a dosage of anti-oxidant for an individual person, wherein the dosage is determined on the basis of an individual factor and a stress index, the individual factor being based on a weight factor of the individual, an age factor of the individual and training factor of the individual based on training history of the individual, and the stress index being based on physical activity of the individual.

2. A method according to claim 1, wherein the dosage varies based on relationships selected from: the weight factor increases with increasing weight of the individual; the age factor increases with increasing age of the individual; the training factor increases as the extent of past physical training of the individual decreases; the individual factor increases as each of the weight factor, the age factor and the training factor increases; the stress index increases as the extent of physical activity increases; the dosage increases as each of the individual factor and the stress index increases; and combinations thereof.

3. A method according to claim 1, wherein the individual factor is the sum of the age factor, the weight factor, and the training factor.

4. A method according to claim 1, wherein the stress index is based on: a physical activity type factor; a training intensity factor; a training time factor; and combinations thereof.

5. A method according to claim 4, wherein the physical activity type factor of a physical activity varies according to relationships selected from: an increase as the physical exertion required to perform the physical activity increases; the training intensity factor increases as the intensity of the individual's performance of a physical activity increases; the training time factor increases as the time spent by the individual performing a physical activity increases; the stress index increases as each of the physical activity type factor, the training intensity factor and the training time factor increases; and combinations thereof.

6. A method according to claim 5, wherein the stress index is determined by multiplying together the physical activity type factor, the training intensity factor and the training time factor.

7. A method according to claim 6, wherein when the individual performs at least two physical activity types and intensities, the stress index is determined by multiplying together the corresponding physical activity type factor, training intensity factor and training time factor for each physical activity type and training intensity combination to obtain a multiplied result, and by then adding together the multiplied results to obtain a total.

8. A method according to claim 1, wherein the age factor is: x if the individual is male and is less than or equal to 30 years of age; x if the individual is female and is less than or equal to 35 years of age; 2x if the individual is male and is greater than or equal to 45 years of age; 2x if the individual is female and is greater than or equal to 55 years of age; and wherein the age factor is 1.5x in any other age category; wherein said factor x is a number.

9. A method according to claim 1, wherein the weight factor is: y if the individual is less than 60 kilograms in weight; 1.5y if the individual is greater than 90 kilograms in weight; and wherein the body weight factor is 1.25y in any other weight category; wherein said factory is a number.

10. A method according to claim 1, wherein the training factor is zero when, in the 3 months immediately prior to performing the method the individual has exercised aerobically at least 5 times per week for at least 30 minutes each time, at a moderate intensity level, and wherein the training factor is 0.5z otherwise, z being a number.

11. A method according to claim 4, wherein:
a first set of physical activity types are assigned a physical activity type factor of a corresponding to a relatively low level of physical exertion required to perform the physical activity types of the set; and,
a second set of physical activity types are assigned a physical activity type factor of 2a corresponding to a relatively moderate level of physical exertion required to perform the physical activity types of the set; and,
a third set of physical activity types are assigned a physical activity type factor of 3a corresponding to a relatively high level of physical exertion required to perform the physical activity types of the set, a being a number.

12. A method according to claim 4, wherein the training time factor for a particular physical activity type and training intensity combination is the number of hours the individual spends per week performing the particular physical activity and training intensity combination.

13. A method according to claim 4,wherein the training intensity factor for a particular physical activity type and training intensity combination is: b if the individual performs the physical activity corresponding to said particular physical activity type and training intensity combination at an easy or low intensity; 2b if the individual performs the physical activity corresponding to said particular physical activity type and training intensity combination at a moderately hard training and moderately intense training level; 3b if the individual performs the physical activity corresponding to said particular physical activity type and training intensity combination at a hard, high intensity training level; b being a number.

14. A method according to claim 1, wherein the dosage of anti-oxidant for the individual is calculated by using the individual factor and the stress index to obtain a corresponding dosage from a nomogram.

15. A method according to claim 1, wherein the dosage is in a number of capsules to be taken by the individual per day.

16. A method according to claim 15, wherein each capsule comprises: 100c and 200c mg of bioflavonoids from pine bark; 50c and 100c mg of Alpha lipoic acid; 50c and 100c mg of Vitamin C and its derivatives; 100c and 200c mg of Vitamin E and its derivatives; wherein c is a positive number.

17. A method according to claim 16, wherein each capsule comprises: 150c mg of bioflavonoids from pine bark; 100c mg of Alpha lipoic acid; 100c mg of a Vitamin C ester; 100c mg of Vitamin E as d-alpha-tocopherol acid succinate; wherein c is a positive number.

18. A method according to claim 16, wherein each capsule comprises: 150c mg of bioflavonoids from pine bark; 50c mg of Alpha lipoic acid; 100c mg of a Vitamin C ester; 150c mg of Vitamin E as d-alpha—tocopherol acid succinate; wherein c is a positive number.

19. A method according to claim 16, wherein c=1.

20. A method of determining a dosage of anti-oxidant for an individual person, wherein the dosage is determined on the basis of an individual factor (IF) and a stress index (SI), and wherein;
IF=WF+AY+TF, and
SI=Sum of {PATF x TIF x TFF} for each possible PATF and TIF combination, and the dosage increases as each of IF and SI increases;
wherein
WF is the weight factor which increases with increasing weight of the individual,
AF is the age factor which increases with increasing age of the individual,
TF is the training factor which increases as the extent of past physical training of the individual decreases,
PATF is a physical activity type factor of a current and future physical activity of the individual and PATF increases as the physical exertion required to perform the corresponding physical activity increases,
TIF is the training intensity factor, where TIF increases as the intensity of the individual's performance of a corresponding physical activity increases, and
TTF is a training time factor for a corresponding PATF and TIF combination, where TTF increases as the time spent by the individual performing a corresponding physical activity increases.

21. An oral formulation for treatment of oxidative stress induced by physical training comprising: between 100c and 450c mg of bioflavonoids from pine bark; 50c and 150c mg of Alpha lipoic acid; 50c and 300c mg of Vitamin C and its derivatives; 100c and 450c mg of Vitamin E and its derivatives; wherein c is a positive number.

22. An oral formulation according to claim 21, wherein said formulation comprises: 150c mg of bioflavonoids from pine bark; 100c mg of Alpha lipoic acid; 100c mg of a Vitamin C ester; 100c mg of Vitamin E as d-alpha—tocopherol acid succinate; c being a positive number.

23. An oral formulation according to claim 21, wherein said formulation comprises: 150c mg of bioflavonoids from pine bark; 50c mg of Alpha lipoic acid; 100c mg of a Vitamin C ester; 150c mg of Vitamin E as d-alpha-tocopherol acid succinate; c being a positive number.

24. An oral formulation according to claim 21, wherein the mixture is in a capsule.

25. An oral formulation according to claim 21, wherein c=1.
